(19) United States
(12) Patent Application Publication
(10) Pub. No.: US 2003/0017493 A1
(43) Pub. Date: Jan. 23, 2003

(54) Methods for the detection and treatment of chronic immune diseases

(76) Inventors: Marc Fremont, Vccle (BE); Patrick Englebienne, Zingem (BE); C.V. Taylor Herst, Oakland, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/174,512

(22) Filed: Jun. 17, 2002

Related U.S. Application Data
(60) Provisional application No. 60/299,829, filed on Jun. 20, 2001.

(51) Int. Cl. C12Q 1/68; G01N 33/567

(52) U.S. Cl. 435/6; 435/7.21

(57) ABSTRACT

Methods are provided for diagnosing/characterizing chronic immune disease activity in a subject. In the subject methods, the extracellular nuclease activity, measured directly and/or indirectly, of the subject is assayed and the resultant value is employed in the diagnosisis/characterization. More specifically, a sample, e.g., plasma or a fraction thereof, is obtained from a subject suspected of having or known to have a chronic immune disease. The sample is then assayed for nuclease activity, either directly or indirectly. The assay results are used to diagnose the presence of chronic immune disease and/or characterize chronic immune disease activity in a subject, and/or to determine appropriate treatments protocols. Also provided by the subject invention are methods of treating chronic immune disease conditions by enhancing extracellular nuclease activity. Also provided by the subject invention are kits for practicing the methods.

### CROSS REFERENCE TO RELATED APPLICATIONS

Pursuant to 35 U.S.C. §119 (e), this application claims priority to the filing date of the U.S. Provisional Patent Application Serial No. 60/299,829 filed Jun. 20, 2001; the disclosure of which are herein incorporated by reference.

### INTRODUCTION

### TECHNICAL FIELD

The field of invention is chronic immune disease, particularly multiple sclerosis and chronic fatigue syndrome.

### BACKGROUND OF THE INVENTION

Chronic immune diseases can be highly debilitating, often requiring treatment. Two such chronic immune diseases are multiple sclerosis and chronic fatigue syndrome.

Multiple sclerosis (MS) is a neurological illness of unknown etiology associated with attacks of focal or multifocal neurological dysfunction indicating lesions within the central nervous system (CNS). In America and Northern Europe, MS is the most common neurological disease, with prevalence rates estimated between 50-100 per 100,000 population. The onset of disease is most common in early adulthood. Recurrent attacks can occur over many years, with approximately 30 percent of the patients progressing to a severe form of the disease that can be fatal.

MS is pleomorphic in its presentation. The clinical manifestations are determined in part by the location of the foci of demyelination within the CNS. Classical features of the disease include impaired vision, nystagmus, dysarthria, ataxia and intention tremor, and weakness/paralysis of one or more limbs. The demyelination is likely due to an autoimmune, inflammatory response that results in the destruction of the myelin sheath covering the axon of the peripheral nerves in the CFS.

The most common form of the disease is episodic. Symptoms develop with subsequent recovery, followed by another attack. In approximately 50 percent of all patients with MS, attacks become more frequent, usually with a worsening of symptomatology. In 30 percent, the disease develops into what is referred to as “progressive/relapsing,” the most severe form of the disease. In this state remissions are rare and patients frequently become wheelchair bound.

The diagnosis of MS remains problematic, and frequently the disease is not diagnosed until the patient has experiences two or more “attacks.” To aid the clinician, the only laboratory test available is testing the cerebrospinal fluid for oligoclonal bands, present in approximately 90 percent of all patients. Examination of the brain for demyelinating plaques, using magnetic resonance imaging (MRI) is useful but expensive and is not warranted except in a small group of patients in which all other clinical and laboratory tests are negative.

There is no diagnostic laboratory test to determine if a patient is having an “attack,” to monitor the progress of the “attack,” to determine if the patient is progressing to a more active form of the disease (i.e., progressive/relapsing), nor is any laboratory test available as a prognostic indicator and/or to monitor therapy if administered.

Chronic Fatigue Syndrome (CFS) is an illness of unknown etiology. CFS is often associated with sudden onset, flu-like symptoms, debilitating fatigue, low-grade fever, myalgia and neurocognitive dysfunction. CFS patients typically display reduced Karnofsky Performance (KPS) scores. The KPS measures an individual's ability to function and carry on normal activities. KPS scores range from zero (0) for a completely non-functional or dead patient to one hundred (100) for a completely normal function.

Diagnosis of CFS remains one of exclusion. An accumulating body of evidence suggests that CFS is associated with dysregulation of both humoral and cellular immunity, including mitogen response, reactivation of viruses, abnormal cytokine production, diminished natural killer cell function and changes in intermediary metabolites.

It has been suggested that the clinical and immunological abnormalities observed in MS and CFS might be caused by defects in the interferon-inducible pathways i.e., the 2′-5′-oligoadenylate (2-5A) synthetase/RNase L and p68 kinase (PKR) antiviral defense pathways (Suhadolnik et al., Clin. Infect. Dis. 18:S96-S104, 1994; Suhadolnik et al., In Vivo 8:599-604, 1994). The 2-5A synthetase/RNase L pathway is part of the antiviral defense mechanism in mammalian cells (Lengyel, Ann. Rev. Biochem. 51:251-282, 1982; Sen et al., Adv. Virus Res. 42:57-102, 1993).

When activated by dsRNA, 2-5A synthetase converts ATP to 2′-5′-linked oligoadenylates with 5′ terminal phosphates. Biologically active 2-5A binds to and activates a latent endoribonuclease, RNase L, which in turn hydrolyzes single-stranded cellular and viral RNA, primarily after UpNp sequences, thereby inhibiting protein synthesis. In addition, circulating white blood cells from patients with CFS have been demonstrated to contain abnormal, low molecular weight forms of RNase L (Suhadolnik et al., J. Interferon & Cytokine Res. 17:377-385, 1997; De Meirleir et al., Am. J. Med. 108:99-105, 2000).

As the above discussion demonstrates, currently employed methods of diagnosing and/or characterizing MS or CFS disease activity in a subject are inadequate. As such, there is a continued need in the field to develop additional means for diagnosing and/or characterizing MS or CFS disease activity in a subject.

### RELEVANT LITERATURE

U.S. Patents of interest include: U.S. Pat. Nos. 5,985,565, 6,020,124, 6,156,504, and 6,207,366. Also of interest is WO 91/00097. Other references of interest include: Komaroff, Am. J. Med. 108:69-71, 2000; Leon et al., Cancer Research 37:646-650, 1977; Steinman, J. Clin. Invest. 73:832-841, 1984; Shapiro et al., Cancer 51:2116-2120, 1983; Sorenson et al., Cancer Epid., Biomarkers & Prevention 3:67-71, 1994; Vasioukhin et al., British J. Hematol. 86:774-779, 1994; Kamm et al., Clin. Chem. 18:519-522, 1972; Stroun et al., Oncology 46:318-322, 1989; Chen et al., Nature Medicine 2:1033-1035, 1996; Nawroz et al., Nature Medicine 2:1035-1037, 1996; Urnovitz et al., Clin. Diag. Lab. Immunol. 6:330-335, 1999; Fournie et al., Cancer Letters 91:221-227, 1995, Durie et al., Blood 90:1090, 1997; Davis et al., Lupus 8:68-76, 1999; Patel et al., Tumour Biol. 21:82-9, 2000; Pan et al., J. Biol. Chem. 273:18374-18381,1998; Prince et al., Clin. Exp. Immunol. 113:289-296, 1998.

### SUMMARY OF THE INVENTION

Methods are provided for diagnosing/characterizing chronic immune disease activity in a subject. In the subject methods, the extracellular nuclease activity, measured directly and/or indirectly, of the subject is assayed and the resultant value is employed in the diagnosisis/characterization. More specifically, a sample, e.g., plasma or a fraction thereof, is obtained from a subject suspected of having or known to have a chronic immune disease. The sample is then assayed for nuclease activity, either directly or indirectly. The assay results are used to diagnose the presence of chronic immune disease and/or characterize chronic immune disease activity in a subject, and/or to determine appropriate treatments protocols. Also provided by the subject invention are methods of treating chronic immune disease conditions by enhancing extracellular nuclease activity. Also provided by the subject invention are kits for practicing the methods.

### BRIEF DESCRIPTION OF THE FIGURES

FIG. 1 represents a densitometric scan of an agarose gel that has been electrophoresed to separate supercoiled plasmid DNA from nicked linearized plasmid DNA to demonstrate the presence or absence of nucleases in the circulation. Included in the description of each lane is the ratio of RNase L fragments as calculated by [Log10((LMW/HMW)*10)] as assayed in PBMC extracts from CFS patients.

### DESCRIPTION OF THE SPECIFIC EMBODIMENTS

Methods are provided for diagnosing and/or characterizing chronic immune disease activity in a subject. In the subject methods, the extracellular nuclease activity is employed in the diagnosis/characterization. In practicing the subject methods, a sample, e.g., plasma or a fraction thereof, is obtained from a subject suspected of having or known to have a chronic immune disease. The sample is then assayed for the nuclease activity, either directly or indirectly. The assay results are used to diagnose the presence of chronic immune disease activity and/or characterize chronic immune disease activity in the subject, e.g. to confirm an initial chronic immune disease diagnosis, to determine the stage of the disease, to monitor disease progression, to predict disease attacks, and the like. In addition, methods of treating chronic immune disease conditions by enhancing extracellular nuclease activity are provided. Also provided by the subject invention are kits for practicing the methods.

Before the invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the invention components which are described in the publications which might be used in connection with the presently described invention.

As summarized above, the subject invention provides a method of diagnosing the presence of a chronic immune disease in a host. In other words, the subject invention provides a means for determining whether a host is suffering from a chronic immune disease. Specifically, the subject invention provides a method of determining whether a host is suffering from MS or CFS. MS and CFS are disease conditions as defined above and throughout the following description.

In determining whether a host suffers from a chronic immune disease, a sample from the host is assayed for the activity of one or more nucleases of interest, where the nucleases may be deoxyribonucleases, ribonucleases, etc., where human nucleases of are of particular interest, where representative nucleases include, but are not limited to: human DNase I-like nuclease, hDNaseI, RNase L, RNase 1, RNase 2, RNase3, RNase 4, angiogenin, RNase 6, and the like. In many embodiments, the nuclease of interest are human deoxyribonucleases, particular circulating (i.e., extra-cellular) human deoxyribonucleases. Information regarding the presence or absence of circulating nucleases, as well as the type of nuclease(s) and/or the amount of nuclease(s), is useful in determining the presence or absence of disease and if present, the level of severity of illness, i.e., in diagnosing and characterizing a chronic immune disease in a host.

As part of the diagnosis, one may also evaluate the subject for other symptoms of the disease of interest to be diagnosed, e.g. the MS or CFS symptoms described in the background section, supra, as well as in other parts of this application.

Also provided by the subject invention are methods of characterizing the chronic immune disease activity, e.g. CFS or MS disease activity, in a subject suspected of having, or known to have, a chronic immune disease, e.g. CFS or MS. Subjects suspected of having, or known to have, a chronic immune disease and thus amenable to the subject methods can be identified using any convenient protocol. One convenient protocol is diagnosis based on clinical symptoms. A number of different clinical symptoms may be used to identify subjects that may have or have the chronic immune disease of interest, where the specific symptoms employed will necessarily depend on the specific chronic immune disease. For example, where the chronic immune disease of interest is CFS, clinical symptoms of interest include: fatigue of six months or longer that causes a reduction in effort of greater than 50 percent of normal output, athralgia, myalgia, sore throat accompanied by swollen glands, cognitive dysfunction (e.g. memory loss); and the like. For MS, clinical symptoms include: weakness of the limbs; sensory symptoms, e.g. paresthesia or hypesthesia; ataxia; optic neuritis; diplopia; trigeminal neuralgia; facial paralysis; vertigo; urinary or bowel movement abnormalities; and cognitive dysfunction, e.g. memory loss, impaired attention, problem-solving difficulties, slowed information processing, and difficulty in shifting between cognitive tasks. The presence of one or more of the above symptoms may be used to identify subjects suspected of suffering from CFS or MS, respectively. Other assays may also be employed, including MRI imaging, the oligoclonal band assay described in greater detail infra, etc.

The first step of the subject methods is to obtain a suitable sample from the subject or patient of interest, i.e. a patient suspected of having or known to have the chronic immune disease of interest, e.g. CFS or MS. Suitable samples are those that can be assayed to determine the amount of extracellular nucleases in the host, particularly circulatory nucleases in the host. The sample is derived from any initial source that contains or may contain extracellular nucleases. Sample sources of interest include, but are not limited to: CSF, urine, saliva, tears, tissue derived samples, e.g. homogenates, and blood or derivatives thereof. In many embodiments, the sample is one that is derived from fluids into which the proteins of interest have been released, e.g. are present. In many embodiments, the sample is a sample in which circulates in the host, e.g., a blood sample. As such, in many embodiments a suitable initial source for the patient sample is blood. As such, the sample employed in the subject assays of these embodiments is generally a blood-derived sample. The blood derived sample may be derived from whole blood or a fraction thereof, e.g. serum, plasma, etc. Of particular interest as a sample source is the extracellular fraction of blood referred to as serum or plasma.

The sample may be assayed the activity of one or more different types of nucleases, including DNases, RNases etc., where human DNases and RNases are of particular interest, where in certain embodiments the nuclease is a human DNase, e.g., DNase I. The sample may be assayed for the nuclease activity(ies) of interest (e.g., DNase and/or RNase activity) using any convenient methodology. The assay employed may be a direct assay or an indirect assay. By direct assay is meant an assay that provides for a direct detection of nuclease activity in the sample, e.g., by measurement of an increase or decrease of degradation of a substrate for the target nuclease relative to a control specimen. By indirect assay is meant an assay that detects the presence or absence (e.g., an assay yielding direct information regarding the presence, type and quantity of nuclease present) a target nuclease and relates this information to the activity of the target nuclease in the sample.

Direct assays to measure the activity of nuclease species may be any one of a number of assays that use a nuclease substrate and measure the activity of the target nuclease(s) by evaluating changes in the amount of nuclease substrate. Any convenient cleavage product detection format may be employed as described supra. Depending on the detection format employed, the source of the nucleic acid substrate may or may not be labeled. For example, one convenient assay employs the use of a nucleic acid substrate having a fluorescent or other detectable label attached to one end of the substrate, the other end attached to a solid support (e.g., bead). The nucleic acid substrate is then contacted with the sample, as described above, and, following incubation, any nucleic acid cleavage products are detected. Non-labeled protocols may also be employed, where the cleavage products are detected with subsequent members of a signal producing system, e.g., labeled antibody. Following detection of the nucleic acid cleavage products, the presence of, and generally amount of nucleic acid cleavage products is related to the nuclease activity of the sample. In other words, the pattern of nucleic acid cleavage products in the reaction is related to the nuclease characteristics or ability of the sample. For example, the presence of nucleic acid cleavage products indicates that the sample comprises the target nuclease activity, while the amount of the cleavage products indicates the level of nuclease activity.

Another representative nuclease activity assay, e.g., direct assay, is that based on the method described in Prince et al., Clin. Exp. Immunol. 113:289-296, 1998. In such assays, the nuclease substrate is typically pre-labeled. The pre-labeled nucleic acid may be single or double stranded deoxyribonucleic acid (ssDNA or dsDNA) or single or double stranded ribonucleic acid (ssRNA or dsRNA) isolated from various sources including but not limited to viruses, bacteria, and eukaryotes. As an example, DNA-specific nuclease activity may be detected and quantified using a ³³P-labeled dsDNA substrate prepared from the addition of single stranded M13 phage DNA template to primers specific for this DNA sequence in the presence of DNA polymerase and alpha-³³P-labeled dATP. The radiolabel is incorporated into the synthesis of the new strand and the result is a dsDNA labeled with 33^{P} This substrate may then be contacted with a patient specimen or derivative thereof, e.g., dilution thereof, and incubated at a temperature to allow nuclease-specific activity to occur. Any nucleases present that are specific for dsDNA will cause the degradation of substrate into free nucleotides that are unable to be precipitated with acid. The resulting degradation of the ³³P-labeled dsDNA can be quantified by measuring the amount of ³³P-labeled dsDNA that can be precipitated from the reaction mixture as compared to a control containing no nuclease and/or a known quantity of nuclease.

Another technique useful in the direct detection of nuclease activity of dsDNA specific nucleases is the ‘nickase’ assay based on the differential migration of supercoiled plasmid DNA as opposed to relaxed plasmid DNA of the same molecular weight. Such assays are known to those of skill in the art. See e.g., Pan et al., J. Biol. Chem. 273:18374-18381, 1998. Due to the supercoiled, super-helical structure of plasmid DNA, such a molecule migrates in an electrical field faster than its calculated molecular weight (i.e., the superhelical structure allows the dsDNA to migrate as if it were ‘smaller’ than it actually is). When such a supercoiled structure comes into contact with a dsDNA-specific nuclease, activity of the nuclease (‘nicking’ the supercoil) will cause the supercoiled structure to relax and unwind. The unwound plasmid subsequently migrates more slowly when subjected to an electric field than the supercoiled plasmid species. Minute amounts of dsDNA-specific nuclease activity can be detected by contacting the patient specimen, or a dilution thereof, with supercoiled plasmid DNA and incubating at a temperature to allow nuclease-specific activity to occur. The resultant relaxed plasmid may be readily detected and quantified by gel electrophoresis (or other suitable method) as compared to a control containing no nuclease and/or a known quantity of nuclease. Various sources of supercoiled plasmid DNA are available and the assay made be made more sensitive by the inclusion of dyes and/or other covalent labeling techniques.

A similar method may be used to quantify ss- or dsRNA-specific nuclease activity. The patient specimen is contacted with a known quantity of ssRNA or dsRNA, depending on the nuclease species to be identified. The ssRNA or dsRNA may be synthetic (i.e. commercially manufactured) or from viruses, bacterial or eukaryotic cells (see Wreshner, et al., Nuc. Acids. Res. 9:1571-1581, 1981, and Fronko, et al., Biochem. Biophys. Res. Commun. 153:448-453, 1988, as is known to those of skill in the art). After incubation of the RNA substrate with the patient specimen, gel electrophoresis may be used to separate the fragments of RNA substrate from the remaining amount of intact (i.e. non-degraded) RNA substrate. Any convenient method of labeling the RNA substrate may be used, such as but not limited to the inclusion of radiolabel or fluorescent dye.

With respect to indirect measures of nuclease activity, e.g., assays which detect the presence, and optionally amount, of one or more nucleases in sample (or even fragments thereof), a number of convenient methods exist for the indirect detection of nuclease species present in a subject sample. Antibodies that specifically bind to the subject nuclease (or target fragment thereof) can be prepared using a variety of convenient methods known to those of skill in the art. See Guide to Protein Purification, supra, as well as Antibodies, A Laboratory Manual (Harlow & Lane eds., Cold Spring Harbor Press, 1988). The antibodies may be polyclonal or monoclonal antibodies depending on the nature of the intended use, as long as they are specific for one or more forms of the nuclease(s) of interest.

For preparation of polyclonal antibodies, the first step is immunization of the host animal with nuclease or an immunogenic fragment, including fragment derivative thereof, where the nuclease immunogen will preferably be in substantially pure form, comprising less than about 1% contaminant. The immunogen may comprise complete nuclease, fragments or derivatives thereof. To increase the immune response of the host animal, the immunogen may be combined with an adjuvant, where suitable adjuvants include alum, dextran, sulfate, large polymeric anions, oil & water emulsions, e.g. Freund's adjuvant, Freund's complete adjuvant, and the like. The immunogen may also be conjugated to synthetic carrier proteins or synthetic antigens. A variety of hosts may be immunized to produce the polyclonal antibodies. Such hosts include rabbits, guinea pigs, rodents, e.g. mice, rats, sheep, goats, and the like. The immunogen is administered to the host, usually intradermally, with an initial dosage followed by one or more, usually at least two, additional booster dosages. Following immunization, the blood from the host is collected, followed by separation of the serum from the blood cells. The Ig present in the resultant antiserum may be further fractionated using known methods, such as ammonium salt fractionation, DEAE chromatography, and the like.

As with the preparation of polyclonal antibodies, the first step in preparing monoclonal antibodies specific for nuclease and fragments thereof is to immunize a suitable host, where suitable hosts include rats, hamsters, mice and the like, and are preferably mice. The nuclease immunogen, which as above, may be the entire nuclease protein or a fragment or derivative thereof, is administered to the host in any convenient manner, where such methods include: subcutaneous injection with adjuvants, nitrocellulose implants comprising the immunogen, intrasplenic injections, and the like, where the immunization protocol may be modulated to obtain a desired type of antibody, e.g. IgG or IgM, where such methods are known in the art. Following immunization, plasma cells are harvested from the immunized host, where sources of plasma cells include the spleen, lymph nodes and the like, with the spleen being preferred. The plasma cells are then immortalized with myeloma cells to produce hybridoma cells. A variety of myeloma cell lines are available and known to those of skill in the art. The plasma and myeloma cells are fused by combining the cells in a fusion medium usually in a ratio of about 10 plasma cells to 1 myeloma cell, where suitable fusion mediums include a fusion agent, e.g. PEG 1000, and the like. Following fusion, the fused cells are selected, e.g. by growing on HAT medium. Following hybridoma cell production, culture supernatant from individual hybridomas is screened for reactivity with nuclease using standard techniques, where such screening techniques include ELISA, dot blot immunoassays and the like. The antibody may be purified from the supernatants or ascites fluid by conventional techniques, e.g. affinity chromatography nuclease bound to an insoluble support, protein A sepharose and the like.

The above prepared or obtained antibodies may be modified in a number of different ways to optimize their utility for use in a particular immunoassay. For example, antibody fragments, such as Fv, F(ab)₂ and Fab may be prepared by cleavage of the intact protein, e.g. by protease or chemical cleavage.

The antibodies, fragments or derivatives thereof may also be labeled in order to facilitate detection. A variety of protein labeling schemes are known in the art and may be employed, the particular scheme and label chosen being the one most convenient for the intended use of the antibody, e.g. immunoassay. Examples of labels include labels that permit both the direct and indirect measurement of the presence of the antibody. Examples of labels that permit direct measurement of the antibody include radiolabels, such as ³H or ¹²⁵I, fluorescers, dyes, beads, chemilumninescers, colloidal particles, and the like. Examples of labels which permit indirect measurement of the presence of the antibody include enzymes where a substrate may provide for a colored or fluorescent product. For example, the antibodies may be labeled with a covalently bound enzyme capable of providing a detectable product signal after addition of suitable substrate. Instead of covalently binding the enzyme to the antibody, the antibody may be modified to comprise a first member of specific binding pair which specifically binds with a second member of the specific binding pair that is conjugated to the enzyme, e.g. the antibody may be covalently bound to biotin and the enzyme conjugate to streptavidin. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art.

In immunoassays of the subject invention, a number of different immunoassay formats are known in the art and may be employed (Prince et al., supra). Representative assay formats include Western blots on protein gels or protein spots on filters, where the antibody is labeled as described above, as is known in the art.

Other immunoassays include those based on competitive formats, as are known in the art. One such format would be where a solid support is coated with nuclease. Labeled antibody is then combined with the patient derived sample suspected to produce a reaction mixture which, following sufficient incubation time for binding complexes to form, is contacted with the solid phase bound nuclease. The amount of labeled antibody which binds to the solid phase will be proportional to the amount of nuclease or fragments thereof in the sample, and the presence of nuclease and fragments thereof may therefore be detected. Other competitive formats that may be employed include those where the sample is combined with a known amount of labeled nuclease and then contacted with a solid support coated with antibody specific for the nuclease of interest. Such assay formats are known in the art and further described in both Guide to Protein Purification, supra, and Antibodies, A Laboratory Manual, supra. Sandwich-format assays may also be employed. A sandwich assay is performed by initially attaching a first of the two types of antibodies to an insoluble surface or support. This first antibody may be bound to the surface by any convenient means, depending upon the nature of the surface, either directly or through specific antibodies. The particular manner of binding is not crucial so long as it is compatible with the reagents and overall methods of the invention. They may be bound to the plates covalently or non-covalently, preferably non-covalently. The insoluble supports may be any compositions to which antibodies or fragments thereof can be bound, which is readily separated from soluble material, and which is otherwise compatible with the overall method of measuring nuclease in the sample. The surface of such supports may be solid or porous and of any convenient shape. Examples of suitable insoluble supports to which the receptor is bound include beads, e.g. magnetic beads, membranes and microtiter plates. These are typically made of glass, plastic (e.g. polystyrene), polysaccharides, nylon or nitrocellulose. Microtiter plates are especially convenient because a large number of assays can be carried out simultaneously, using small amounts of reagents and samples. Before adding patient samples or fractions thereof, the non-specific binding sites on the insoluble support i.e. those not occupied by the first antibody, are generally blocked. Preferred blocking agents include non-interfering proteins such as bovine serum albumin, casein, gelatin, and the like. Alternatively, several detergents at non-interfering concentrations, such as Tween, NP40, TX100, and the like may be used. Samples, fractions or aliquots thereof are then added to separately assayable supports (for example, separate wells of a microtiter plate) containing support-bound allergen. Preferably, a series of standards, containing known concentrations of nuclease is assayed in parallel with the samples or aliquots thereof to serve as controls. Generally from about 0.001 to 1 ml of sample, diluted or otherwise, is sufficient, usually about 0.01 ml sufficing. Preferably, each sample and standard will be added to multiple wells so that mean values can be obtained for each. The incubation time should be sufficient for nuclease molecules to bind the insoluble first antibody. Generally, from about 0.1 to 3 hr is sufficient, usually 1 hr sufficing. After incubation, the insoluble support is generally washed of non-bound components. Generally, a dilute non-ionic detergent medium at an appropriate pH, generally 7-8, is used as a wash medium. From one to six washes may be employed, with sufficient volume to thoroughly wash nonspecifically bound proteins present in the sample. After washing, a solution containing the second nuclease or nuclease fragment specific antibody is applied. The second antibody may be labeled, as described above, to facilitate direct, or indirect detection and/or quantification of binding. Examples of labels which permit direct measurement of immunocomplexes include radiolabels, such as ³H or ¹²⁵I, fluorescers, dyes, beads, chemilumninescers, colloidal particles, and the like. Examples of labels which permit indirect measurement of binding include enzymes where the substrate may provide for a colored or fluorescent product. In a preferred embodiment, the second antibody is labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. Alternatively, the antibody may be unlabeled. In this case, a labeled second receptor-specific compound is employed which binds to the second antibody. Such a second receptor-specific compound can be labeled in any of the above manners. It is possible to select such compounds such that multiple compounds bind each molecule of bound second receptor. Examples of second antibody/second receptor-specific molecule pairs include antibody/anti-antibody and avidin (or streptavidin)/biotin. Since the resultant signal is thus amplified, this technique may be advantageous where only a small amount of nuclease or fragment thereof is present. An example is the use of a labeled antibody specific to the second antibody. The volume, composition and concentration of second antibody solution provides for measurable binding to the nuclease already bound to the first antibody. Generally, the same volume as that of the sample is used: from about 0.001 to 1 ml is sufficient, usually about 0.1 ml sufficing. The concentration will generally be sufficient to saturate all nuclease potentially bound to first antibody. The concentration generally will be about 0.1 to 50 μg/ml, preferably about 1 μg/ml. The solution containing the second antibody is generally buffered in the range of about pH 6.5-9.5. The solution may also contain an innocuous protein as previously described. The incubation time should be sufficient for the labeled ligand to bind available molecules. Generally, from about 0.1 to 3 hr is sufficient, usually 1 hr sufficing. After the second antibody has bound, the insoluble support is generally again washed free of non-specifically bound second receptor, essentially as described for prior washes. After non-specifically bound material has been cleared, the signal produced by the bound conjugate is detected by conventional means. Where an enzyme conjugate is used, an appropriate enzyme substrate is provided so a detectable product is formed. More specifically, where a peroxidase is the selected enzyme conjugate, a preferred substrate combination is H₂O₂ and O-phenylenediamine that yields a colored product under appropriate reaction conditions. Appropriate substrates for other enzyme conjugates such as those disclosed above are known to those skilled in the art. Suitable reaction conditions as well as means for detecting the various useful conjugates or their products are also known to those skilled in the art. For the product of the substrate 0-phenylenediamine for example, light absorbance at 490-495 nm is conveniently measured with a spectrophotometer.

Depending on the particular nature of the antibody based assay employed, it may be desirable to employ antibodies that are capable of distinguishing between the various nuclease forms and/or fragment forms. For example, in a Western blot assay a single type of antibody that recognizes all of the various nucleases of interest (or all of the native and fragment forms of a nuclease) may be employed, e.g., where the various nuclease species and fragments thereof are pre-separated, e.g. by gel electrophoresis. However, where the various species are not separated prior to detection, e.g. in the competitive and sandwich assays described above, it is desirable to use a plurality of antibodies which are capable of specifically recognizing only a single nuclease species of interest, with substantially no cross-reactivity with other nuclease species that may be present in the sample.

In the subject indirect methods, the sample or fractions thereof are at least assayed for the presence or absence of nuclease species, where the assay may be a direct assay or an indirect assay as indicated above. In some embodiments, qualitative results are sufficient. Thus, one may be interested in identifying the presence or absence of nucleases as a marker for the chronic immune disease, e.g. in the diagnostic methods described above. Alternatively, one may be interested in making a quantitative determination of the amount of nuclease(s) present. In many embodiments, the assays employed provide at least semi-quantitative detection of the various nuclease species, and not just qualitative detection.

In many embodiments, based on the results of the direct and/or indirect assays of nuclease activity, e.g., presence or absence of the various nuclease species, and usually the semi-quantitative values obtained for each of the species of interest, the chronic immune disease activity in the subject from which the sample was derived is characterized. This broad category of embodiments includes those in which the nuclease species are directly assayed, e.g., those methods where the relative activity of the various nuclease species present is used to characterize the disease; as well as those embodiments in which the nuclease species are indirectly assayed, e.g., where the simple presence or absence of nuclease species is used to characterize the disease; where the relative amount of various nuclease species present is used to characterize the disease; etc.

Characterization of chronic immune disease activity according to the subject methods typically involves comparing the results obtained to a table or other source of predetermined values or reference values which provide information about the disease activity in the host, e.g. that positively or negatively correlate to the presence of the chronic immune disease, a particular stage of the chronic immune disease, and the like. For example, a table of values may be consulted in this step, where the table comprises representative values for the type and amount of nuclease species present as found in patients suffering from the chronic immune disease of interest. The values may be presented in numerical form, in picture form (e.g. as bands on a gel), and the like. By comparing the observed values with these reference values, e.g. by comparing a pattern of the nuclease species in the sample to a reference pattern or picture, characterization of the disease activity, e.g. confirmation of diagnosis, determination of disease state, etc., is readily made. In other embodiments, the ratio of two or more of the different species of nucleases present is then compared to reference list of ratios to characterize the chronic immune disease-activity.

As summarized above, the subject methods are methods of characterizing chronic immune disease activity in a host. The term characterizing is used broadly to refer to derivation of any type of information about the state of the chronic immune disease in the host. As such, the subject methods may be used to confirm an initial diagnosis of chronic immune disease, to determine the state of the disease in a patient known to have the chronic immune disease, to monitor the progression of the disease, to predict the occurrence of an attack, and the like. Where the subject invention is employed to confirm an initial diagnosis, a sample is obtained from subject suspected of having the chronic immune disease (where the subject may be identified as described supra). For example, the sample is assayed for the presence of multiple nuclease species, a ratio of any two species is derived and then compared to reference values, where the reference values correlate given ratios to the presence or absence of the chronic immune disease.

The subject methods are also employed to determine the stage of the chronic immune disease in the subject. In other words, the subject chronic immune disease activity characterization methods may be employed to determine whether the patient is in a remission stage, a chronic stage etc. For example, the subject methods may be employed to determine whether an MS patient is in the relapsing-remitting stage or in the chronic progressive stage of the disease. To determine the stage of the disease, the observed values for the one or more nuclease species, and ratios where desired, in the assayed sample are compared to reference values that are correlated to a particular stage of chronic immune disease, e.g. remitting relapsing or chronic progressive stage of MS.

In yet other embodiments, characterization of disease activity yields information concerning the disease progression in the patient, e.g. whether disease progression has accelerated or slowed. For example, the initial characterization date, i.e. the amount of nuclease species specific activity present in the patient derived sample, could be employed as a baseline value to evaluate subsequent samplings, e.g. at some time following the initial testing, e.g. 3 months. If the amount of nuclease species-specific activity increases in subsequent testing, this indicates that the disease is not progressing and/or may be in remission. Alternatively, if the amount of nuclease species-specific activity decreases, this indicates that the disease is not resolving and/or progressing in severity.

The characterization data obtained from the subject methods may also be used to determine whether a particular therapeutic regimen is having positive affects with respect to the progression of the disease. For example, at various time periods during the course of treatment, the subject methods may be performed to obtain a reading of the amount, type and relative activity of the nuclease species of interest. If the amount of nuclease activity is increasing, this indicates that the treatment regimen is working. Alternatively, if the amount of nuclease activity is decreasing, this indicates that the treatment regimen is not having the desired effect.

In yet other embodiments, the levels of nuclease species activity present in the patient sample may act as an indicator to determine the amount of a specific therapeutic to administer. In particular when such therapeutic regimens are to include a nucleic acid-based therapeutic (e.g., antisense DNA, dsRNA and the like), quantification of nuclease species activity may allow for the dosage of the therapeutic to be adjusted so as to achieve the desired benefit. For example, if the level of nuclease species activity is at low levels, less therapy may need to be administered to achieve a benefit. Alternatively, if the levels of nuclease species activity are increasing, an increasing dose must be administered to overcome the effects of increased nuclease-dependent degradation of the therapeutic. In this embodiment, values obtained from the patient sample are compared to specific reference values before and during the administration of therapy to obtain the correct amount for dosage.

In yet other embodiments, the characterization data obtained from the subject methods is used to predict when a chronic immune disease attack, e.g. MS attack, may occur. In this embodiment, the characterization data is compared to reference values, where some of the reference values correlate to the occurrence of an attack.

In another embodiment, the direct or indirect measure of nuclease activity, e.g., the presence and quantification of nuclease species activity present in a random sample of blood, may be useful as an indicator of the presence or absence of covert infection and/or chronic immune disease. By examining specimens of donated blood (i.e., blood bank specimens) for the presence of nuclease species activity and rejecting any samples that exceed a reference value, a reduction in the number of transfusion-related chronic immune diseases can be effected. In this embodiment values obtained from the patient sample are compared to specific reference values before accepting or rejecting a specimen of blood for transfusion.

Depending on the particular test protocol, the subject methods may further include one or more additional assays associated with the chronic immune disease of interest. For example, one may couple the subject methods with assays that look for the presence of low molecular weight proteins that exhibit RNase L activity, the ratio of high to low molecular weight proteins that exhibit RNase L activity, etc., as described in U.S. Pat. Nos. 5,985,565 and 6,207,366, the disclosures of which are herein incorporated by reference. Other representative assays of interest include biochemical assays capable of identifying MS activity in the subject, e.g. assays which detect the presence of oligoclonal bands in cerebral spinal fluid (CSF). A variety of such assays are known to those of skill in the art and may be employed in the subject methods. See e.g. Lasne et al., J. Neurochem. 36:1872-1875,1981; Mehta, et al., J. Clin. Lab. Immunol. 6:17-22, 1981; Mehta et al., Electrophoresis 9:126-128, 1998; Mehta et al., J. Neuroscience Methods 6:277-282, 1986; Trbojevic-Cepe et al., Neurologija. 38:11-21, 1989.

Also provided by the subject invention are kits for use in carrying out the subject methods. The kits at least comprise reagents necessary for carrying out the nuclease species detection assays, where such kits may include: substrates for use in nuclease activity assays; nuclease specific antibodies and/or immunoassay devices comprising the same; members of a signal producing system, such as antibodies, enzyme substrates, and the like; various buffers for use in carrying out the subject detection assays; and the like. The kits may further include one or more reagents necessary for preparation of the patient derived sample. In addition, the subject kits may further include one or more components employed in fractionation of the sample, such as an electrophoretic medium or precursors thereof, e.g. dried precursors of agarose and/or polyacrylamide gels, one or more buffer mediums or components thereof, and the like. In most embodiments, the kits further include at least an information storage and presentation medium that contains reference data with which assay results may be compared in order to diagnose and/or characterize the chronic immune disease activity in the subject being assayed, i.e. reference data that includes various values of the relative activity of the nuclease species and relates these values to the presence or absence of chronic immune disease and/or the activity of the disease in the host. The information storage and presentation medium may be in any convenient form, such as a printed information on a package insert, an electronic file present on an electronic storage medium, e.g. a magnetic disk, CD-ROM, and the like. In yet other embodiments, the kits may include alternative means for obtaining reference data, e.g. a website for obtaining the reference data “on-line.” The kits may further include means for obtaining the patient sample, e.g. a syringe. The subject kits further typically include instructions for carrying out the subject methods, where these instructions may be present on a package insert and/or the packaging of the kit. Finally, the kit may further include one or more reagents from an additional biochemical assay that is used to detect the presence of and/or characterize the chronic immune disease of interest. For example, where MS is the chronic immune disease of interest, the kits may further include one or more reagents from an assay designed to detect the presence of oligoclonal bands in CSF, e.g. immunoxification reagents (e.g. anti-IgG); labeling reagents, such as silver salts, and the like.

As summarized above, the subject invention also provides methods for treating a host suffering from a chronic immune disease. Specifically, the subject invention provides methods of treating a host suffering from MS or CFS.

In practicing the subject methods, an effective amount of an agent that enhances nuclease activity, particularly an agent that enhances circulatory nuclease activity, is administered to the host suffering from the chronic immune disease. By enhance is meant that the target nuclease activity, in the host, particularly in the circulation of the host, is increased by at least about 2 fold, usually by at least about 3 fold and more usually by at least about 5 fold, as compared to that observed in a control, i.e., serum from the host that has not been contacted by, the active agent(s).

Enhancement of nuclease activity can be accomplished in any convenient manner, where the mode of nuclease activity enhancement typically comprises administering an agent or agents that modulate one or more of the pathways as described below. Particular active agents of interest include, but are not limited to: proteins (recombinant or native) and/or fragments of proteins (recombinant or native) that have nuclease-containing activity; nuclease-specific proteolysis inhibitors(i.e., the specific degradation of nucleases by proteases); and nuclease expression enhancing agents. Each of these types of agents is now described separately in greater detail.

Enhancement of nuclease species-specific activity in the host, particularly in the circulation of the host, may be accomplished by the direct administration of specific nucleases, either in recombinant form or native. Preparations of purified nucleases, including human nucleases, e.g., human DNase I, human ribonucleases etc., are known. See e.g., U.S. Pat. No. 5,830,744, the disclosure of which is herein incorporated by reference. Alternatively, the nuclease may be recombinantly produced using the nucleotide sequence of nuclease of interest, using techniques known to those of skill in the art. The nucleic acid sequence of a variety of different nucleases, including human nucleases, are known. See e.g., U.S. Pat. No. 5,830,744; the disclosure of which is herein incorporated by reference, as well as those sequences having GENBANK Accession nos.: L40817 (DNase I-like), M55983 (hDNaseI), etc. The protein, or active portion thereof, may be introduced into the tissues, circulation, or host cells by any number of routes, including oral, intradermal, intramuscular, intravenous, subcutaneous, or by inhalation. See also the protocols for administered protein therapeutic agents described below.

Also of interest are nuclease cleavage-inhibitory agents of interest for use in the subject methods are agents that inhibit cleavage or fragmentation of nuclease species present in the circulation. The target molecule is a protein or activity, e.g., enzyme, that cleaves native nuclease into fragments and in so doing inactivates the ability of the nuclease to cleave nucleic acid. By inhibit is meant that these agents at least reduce, if not substantially or complete stop, the cleavage of one or all nuclease species present in a sample. Nuclease cleavage-inhibitory agents typically reduce the cleavage of nuclease species by at least about 2 fold, usually at least about 3 fold and more usually at least about 5 fold.

Inhibitors of interest include agents that bind to the target molecule (e.g., protease) and concomitantly reduce its activity, as well as agents that reduce the expression of the target molecule so that the overall cleavage activity of the target molecule is reduced. As such, agents of interest include small molecule agents, as may be identified in the assays described below and antibodies specific to inhibiting the action of the nuclease-cleaving target molecules. Small molecule agents of interest include small organic compounds having a molecular weight of more than 50 and less than about 2,500 daltons. The small molecule agents comprise functional groups necessary for structural interaction with proteins, particularly hydrogen bonding, and typically include at least an amine, carbonyl, hydroxyl or carboxyl group, preferably at least two of the functional chemical groups. The small molecule agents often comprise cyclical carbon or heterocyclic structures and/or aromatic or polyaromatic structures substituted with one or more of the above functional groups. Small molecule agents of interest are also found among biomolecules including, but not limited to: peptides, saccharides, fatty acids, steroids, purines, pyrimidines, derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. New potential therapeutic agents may also be created using methods such as rational drug design or computer modeling. Protease specific antibodies may be readily produced using the procedures described above.

In yet other embodiments of the invention, the active agent is an agent that modulates, and generally decreases or down regulates, the expression of the nuclease-specific protease gene in the host. Antisense molecules can be used to down-regulate expression of genes in cells. The anti-sense reagent may be antisense oligonucleotides (ODN), particularly synthetic ODN having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al., Nature Biotechnol. 14:840-844, 1996)

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1993), supra, and Milligan et al., supra.) Preferred oligonucleotides are chemically modified from the native phosphodiester structure, in order to increase their intracellular stability and binding affinity. A number of such modifications that alter the chemistry of the backbone, sugars or heterocyclic bases have been described in the literature.

Among the useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH₂-5′-phosphonate and 3′-NH-5′-O-phosphoroamidate. Peptide nucleic acids replace the entire ribose phosphodiester backbone with a peptide linkage. Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

As an alternative to anti-sense inhibitors, catalytic nucleic acid compounds, e.g. ribozymes, anti-sense conjugates, etc. may be used to inhibit gene expression. Ribozymes may be synthesized in vitro and administered to the patient, or may be encoded on an expression vector, from which the ribozyme is synthesized in the targeted cell (for example, see International patent application WO 9523225, and Beigelman et al., Nucl. Acids Res. 23:4434-4442, 1995). Examples of oligonucleotides with catalytic activity are described in WO 9506764. Conjugates of anti-sense ODN with a metal complex, e.g. terpyridylCu(II), capable of mediating mRNA hydrolysis are described in Bashkin et al., Appl. Biochem. Biotechnol. 54:43-56, 1995.

A further alternative to the above is the use of double-stranded RNA sequences, or the production thereof by introducing vectors for such in the host, the nucleic acid sequences of which are identical to all or part of the nuclease-specific protease gene. Such a double-stranded RNA is capable of binding to and causing the degradation of the homologous mRNA species. Thus, the mRNA coding for the production of nuclease-specific protease is targeted for removal by this method. This technique is referred to as RNA interference, examples of which are described in Tuschl et al., Genes and Development 13:3191-3197, 1999, and Zamore, Cell 101:25-33, 2000.

In yet other embodiments of the subject invention, the active agent is a nuclease expression enhancing agent. By nuclease expression enhancing agent is meant an agent that enhances expression of native nuclease species mRNA or the production of native nuclease protein(s) in the host. Agents of interest include, but are not limited to: nuclease-encoding nucleic acids and protein therapeutic compositions. In this embodiment, the genes or gene fragments are useful in gene therapy to enhance nuclease gene activity. Expression vectors may be used to introduce the nuclease species-specific gene(s) into a cell. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g., plasmid; retrovirus, e.g. lentivirus; adenovirus; and the like, where the vectors are able to transiently or stably be maintained in the cells, usually for a period of at least about one day, more usually for a period of at least about several days to several weeks.

The gene or protein may be introduced into tissues or host cells by any number of routes, including viral infection, microinjection, or fusion of vesicles. Jet injection may also be used for intramuscular administration, as described by Furth et al., Anal. Biochem. 205:365-368, 1992. The DNA may be coated onto gold microparticles, and delivered intradermally by a particle bombardment device, or “gene gun” as described in the literature (see, for example, Tang et al., Nature 356:152-154, 1992), where gold microprojectiles are coated with the DNA and then bombarded into skin cells.

Also of interest is the introduction of nuclease-encoding gene sequences, the protein product of which may be constitutively produced but whose nuclease-encoding action may be specifically activated by the in vivo administration of an activator molecule. An example of such a nuclease-encoding gene sequence would be human RNase L, referenced supra. Recombinant RNase L (rRNase L) remains inactive until it binds to its activator molecule, trimeric oligoadenylate (2′5′A). Upon binding, the rRNase L dimerizes and is activated to degrade any free-floating RNA species in the circulation. Thus the nuclease activity of RNase L could be specifically activated by the administration of 2-5A. The activator molecule may be administered by any of the routes as described supra.

Also of interest is the use of agents that modulate the endogenous nuclease gene(s) of the host to enhance expression. For example, any one of a number of endogenous nuclease gene sequences in a cell can be regulated by an exogenous regulatory sequence inserted into the genome of the cell at location sufficient to at least enhance expression of the specific nuclease gene in the cell. The regulatory sequence may be designed to integrate into the genome via homologous recombination, as disclosed in U.S. Pat. Nos. 5,641,670 and 5,733,761, the disclosures of which are herein incorporated by reference, or may be designed to integrate into the genome via nonhomologous recombination, as described in WO 99/15650, the disclosure of which is herein incorporated by reference. As such, also encompassed in the subject invention is the enhancement of nuclease expression without manipulation of the encoding nucleic acid itself, but instead through integration of a regulatory sequence into the genome of cell of the host that already includes a gene encoding the desired protein, as described in the above incorporated patent documents.

As mentioned above, in the subject methods an effective amount of one or more of the above described active agents is administered to the host, where “effective amount” means a dosage sufficient to produce a desired result, where the desired result is at least an amelioration, if not complete cessation of the chronic immune disease symptoms.

In the subject methods, the active agent(s) may be administered to the host using any convenient means capable of resulting in the desired treatment. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of the agents can be achieved in various ways, including oral, buccal, rectal, parenteral, intraperitoneal, intradermal, transdermal, intracheal, etc., administration.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Where the agent is a polypeptide, polynucleotide, analog or mimetic thereof, e.g. antisense composition, it may be introduced into tissues or host cells by any number of routes, including viral infection, microinjection, or fusion of vesicles. Jet injection may also be used for intramuscular administration, as described by Furth et al., supra. The agent may be coated onto gold microparticles, and delivered intradermally by a particle bombardment device, or “gene gun” as described in the literature as described by Tang et al., supra.

Those of skill in the art will readily appreciate that dose levels can vary as a function of the specific compound, the severity of the symptoms and the susceptibility of the subject to side effects. Preferred dosages for a given compound are readily determinable by those of skill in the art by a variety of means.

As mentioned above, by treatment is meant that at least an amelioration of the symptoms associated with the chronic immune disease, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g. prevented from happening, or stopped, e.g. terminated, such that the host no longer suffers from the condition, or at least the symptoms that characterize the chronic immune disease condition.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

Kits with unit doses of the active agent, usually in oral or injectable doses, are provided. In such kits, in addition to the containers containing the unit doses will be an informational package insert describing the use and attendant benefits of the drugs in treating pathological condition of interest. Preferred compounds and unit doses are those described herein above.

The following examples are offered by way of illustration and not by way of limitation.

### Experimental Section

I. Analysis and Quantification of Low and High Molecular Weight RNase L Protein Species Present in Cell Extracts as Compared to Nuclease Activity Present in Serum from CFS Patients and Healthy Controls

Study subjects were 5 patients who had previously been diagnosed as fulfilling the diagnostic criteria for CFS, and 1 healthy control. At the time of blood sampling, patient symptoms were evaluated and recorded.

A. Procedures

1. Blood Collection, Cell Isolation and Protein Extraction

Blood was collected by venipuncture with and without anticoagulant. The blood without anticoagulant was allowed to clot for one hour at room temperature. Serum was then spun off of the clot, collected, and frozen at −70C. until analysis for nuclease activity. Peripheral blood mononuclear cells (PBMCs) were separated from heparinized blood (30 mLs) by Ficoll-Hypaque density gradient centrifugation. The blood was layered onto 20 mLs of Ficoll-Hypaque (Boyum, Scandinavian Journal of Clinical Laboratory Investigation, 97:101-109, 1968) at a density of 1.077 g/mL at 20C. and centrifuged for 30 minutes at 500×g. The PBMC layer was removed and washed once with 5 volumes of phosphate buffered saline (PBS). The cells were then resuspended in 5 mLs of red blood cell lysing buffer (155 mM NH₄Cl, 10 mM NaHCO₃, 0.1 mM EDTA, pH 7.4), kept on ice for 5 minutes, then centrifuged for 5 minutes at 500×g. The resultant cell pellet was washed once with 15 mLs of PBS and centrifuged for 5 minutes at 500×g. The resultant pellet was then stored at −70C. until the protein extraction procedure could be performed.

To extract the proteins from the cell pellet, PBMCs were resuspended in a volume approximately 5-10 times the packed cell volume in the extract buffer (10 mM HEPES, pH 7.6, 90 mM KCl, 1.5 mM Mg(OAc)₂, 0.5% non-ionic detergent (such as Nonidet P-40 or Igepal CA-630, Sigma Chemical Corporation)). The extract buffer also contained a mixture of protease inhibitors to help stabilize the extract and impeded the action of proteases. Once such commercially available mixture is the MiniComplete protease inhibitor cocktail (Boehringer-Mannheim). This contains aprotinin, leupeptin, pefabloc-SC and EDTA.

The extraction procedure was performed at 2-4 degrees C., holding the cell pellet-extraction buffer in ice water or on wet ice for 5 minutes. The cell pellet-buffer mix was then vortexed at medium speed for 2 minutes at room temperature to ensure complete solubilization of the cell membranes. The cell pellet-buffer mix was then placed at 2-4C. for an additional 5 minutes. The final step was to centrifuge the cell pellet-buffer mix at high speed in a microcentrifuge (16,000×g) for 2 minutes. The supernatant containing the proteins of interest was collected and the cell pellet is discarded. All cell extracts were stored at −70C. until further analysis could be performed.

Quantification of protein in the patient cell extracts was performed using a standard commercially available procedure of a modified Bradford method (Bio-Rad Laboratories) following the manufacturer's recommended procedure.

2. Quantification of 2′-5′A Binding Proteins

Analysis of LMW and HMW RNase L proteins was performed using a radiolabeled 2′-5′A trimer and SDS-PAGE as described by the method of Charachon et al. (Biochemistry 29:2550-2556, 1990). Briefly, 2′-5′A trimer was radiolabeled by the ligation of ³²P-pCp to the 3′ end (method of Charachon). After removal of the 3′ terminal phosphate by treatment with bacterial alkaline phosphatase, the 3′ ribose residue of pC was oxidized with sodium metaperiodate (10 mM final concentration, pH 4.75) for one hour at 4C. to form 2′5′A-³²pC-OX. This reaction mixture was subsequently equilibrated to pH 8.0 by the addition of NaOH. This oxidized molecule was used as the radiolabel in all subsequent reactions for Nuclease protein analysis (referred to below as radiolabeled 2′5′A).

The radiolabeled 2′5′A was incubated with 200 micrograms of cell extract at 2-4C. for 15 minutes to allow the radiolabeled 2′5′A to interact with any 2′5′A-binding proteins present, such as RNase L (all molecular weight species). The 2′-5′A radiolabel was then covalently attached to all RNase L species present by the addition of cyanoborohydride (20 mM in 100 mM phosphate buffer, pH 8.0). The reduction reaction was allowed to occur for 20 minutes at room temperature. SDS-PAGE sample buffer, including a tracking dye, was added to the samples and all samples were incubated at 95C. for 5 minutes to reduce any disulfide bonds present.

The samples were then subjected to standard SDS-polyacrylamide gel electrophoresis using a 4 percent stacking gel and a 10 percent separating gel (Bisbal et al, European Journal of Biochemistry 179:595-602, 1989). Also included in the first lane of each gel was a molecular weight marker, pre-stained to be visible as it migrated during the course of electrophoresis (Bio-Rad Laboratories). The gel was electrophoresed until the tracking dye had migrated to the bottom of the gel (approximately 5 hours at a constant current of 30 mAmps). The gel was then dried and subjected to autoiradiography (Bio-Rad Laboratories FX Imager).

The autoradiographs were then analyzed by densitometry, and quantification of any and all RNase L species present was performed using specialized software (Quantity One from Bio-Rad Laboratories). The results are expressed as the density (or relative amount) of 37 kDa LMW RNase L present divided by the density (or relative amount) of 80 kDa HMW RNase L present, multiplied by a constant factor of 10.

3. Quantification of Nuclease Activity by Digestion of Endogenous DNA

Briefly, the procedure used is as follows: Plasmid DNA was isolated from stationary growth cultures of E.coli by standard procedures and banded on a cesium chloride gradient to isolate and purify the supercoiled fraction (see Sambrook, et al., Cloning, A Laboratory Manual, Cold Spring Harbor Press, 1997). One microgram of supercoiled plasmid in one microliter of Tris EDTA buffer (10 mM Tris, 1 mM EDTA, pH 8.0) was diluted with 23 microliters of dilution buffer containing 10 mM HEPES, pH 7.0, and 2 mM CaCl₂. Twelve microliters of this mixture was incubated with two microliters of patient serum that had been previously diluted, also in dilution buffer, to a dilution of 1:20. This reaction mixture was incubated at 37C. for 15 minutes. Two microliters of agarose gel loading dye (50 percent glycerol and 0.1 percent (w/v) bromphenol blue in water) were added and the sample was immediately loaded into a 0.8 percent agarose gel in 1×Tris Borate EDTA (TBE) buffer, pH 8.0, containing 0.01 percent ethidium bromide and electrophoresed in 1×TBE buffer, being subjected to a voltage of 5 volts per cm of gel for one hour. The gel was visualized using an ultraviolet light. The amount of supercoiled plasmid DNA remaining in the sample, as well as the amount of ‘nicked’ or relaxed plasmid present was determined by densitometric scanning.

B. Analysis of Results

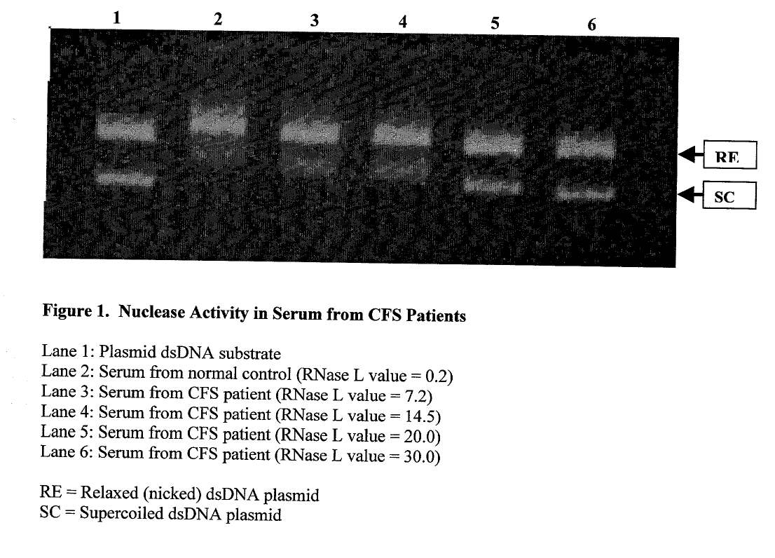

FIG. 1 represents a densitometric scan of an agarose gel that has been electrophoresed to separate supercoiled plasmid DNA (indicated as ‘SC’) from nicked or relaxed plasmid DNA (indicated as ‘RE’). In the experiment represented in FIG. 1, sera from patients previously diagnosed with CFS as based on clinical criteria and as determined by the results of analysis of RNase L fragments, was reacted with supercoiled dsDNA plasmid to measure the amount of nuclease activity present.

From FIG. 1, it is clear that as the levels of RNase L fragmentation increase (i.e., as the ratio of LMW to HMW species of RNase L protein becomes greater, as calculated by [Log 10((LMW/HMW)* 10)]), there is a concomitant decrease in the amount of nuclease species-specific activity, in this case a decrease in dsDNA-specific nuclease activity. At the highest ratio of RNase L LMW to HMW (FIG. 1, Lane 6), the amount of activity of nucleases specific for dsDNA has decreased to a level that is undetectable when compared to dsDNA plasmid alone in the presence of buffer (FIG. 1, Lane 1).

It is evident from the above results and discussion that relatively simple and rapid methods for diagnosing and/or characterizing chronic immune disease (e.g. MS or CFS) activity in a subject are provided by the subject invention. With the subject methods, accurate diagnosis of the chronic immune disease condition, as well the identification of the stage and/or progression of the chronic immune disease condition, may be obtained. As such, the subject methods provide for more accurate diagnostic and/or treatment regimens. In addition, methods of treating hosts for chronic immune disease are provided. Accordingly, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method for diagnosing whether a host suffers from a chronic immune disease, said method comprising:
assaying a sample from said host for the activity of at least one nuclease species to obtain assay results; and
determining whether said host suffers from a chronic immune disease using said assay results;
whereby said host is diagnosed for said chronic immune disease.

2. The method according to claim 1, wherein said chronic immune disease is selected from the group consisting of CFS and MS.

3. The method according to claim 1, wherein said sample is a blood derived sample.

4. The method according to claim 1, wherein said blood derived sample is derived from serum.

5. The method according to claim 1, wherein said host is a human.

6. A method of characterizing chronic immune disease activity in a human subject, said method comprising:
(a) obtaining a sample from said subject;
(b) determining the nuclease activity of one or more nuclease species present in said sample; and
(c) using said results to characterize the chronic immune disease activity in said subject.

7. The method according to claim 6, wherein said chronic immune disease is selected from the group consisting of CFS and MS.

8. The method according to claim 6, wherein said sample is a blood derived sample.

9. The method according to claim 6, wherein said blood derived sample is derived from serum.

10. The method according to claim 6, wherein said method is a method of confirming whether said subject suffers from said chronic immune disease.

11. A method of characterizing a chronic immune disease activity in a human subject, said method comprising:
(a) obtaining an acellular circulatory sample from said subject;
(b) determining the activity of one or more nucleases in said sample to obtain assay results; and
(c) using said results to characterize said chronic immune disease activity in said subject.

12. The method according to claim 11, wherein said chronic immune disease is selected from the group consisting of CFS and MS.

13. The method according to claim 11, wherein said sample is serum.

14. The method according to claim 11, where said sample is plasma.

15. A kit for use in characterizing a chronic immune disease activity in a subject, said kit comprising:
(a) means for assaying a sample for the activity of at least one nuclease species; and
(b) a medium comprising reference information relating the activity of one or more nuclease species to chronic immune disease activity.

16. The method according to claim 15, wherein said chronic immune disease is selected from the group consisting of CFS and MS.

17. The kit according to claim 15, wherein said kit further comprises means for obtaining a sample from said subject.

18. The kit according to claim 15, wherein said kit further comprises instructions for practicing the method of claim 1.

19. The kit according to claim 15, wherein said means comprises a substrate for said at least one nuclease.

20. A method for treating a host suffering from a chronic immune disease, said method comprising:
administering to said host an effective amount of an agent that enhances extracellular nuclease activity in said host.

21. The method according to claim 20, wherein said chronic immune disease is selected from the group consisting of CFS and MS.

22. The method according to claim 20, wherein said agent is a nuclease.

23. The method according to claim 20, wherein said agent is a nuclease cleavage-antagonist.

24. The method according to claim 20, wherein said agent enhances nuclease expression in said host.

25. The method according to claim 20, wherein said host is a mammal.

26. The method according to claim 25, wherein said mammal is a human.
